(19) United States
(12) Patent Application Publication
(10) Pub. No.: US 2026/0083974 A1
(43) Pub. Date: Mar. 26, 2026

(54) IMPLANTABLE ELECTRICAL CONTACT ARRANGEMENT

(71) Applicant: Neuroloop GmbH, Freiburg (DE)

(72) Inventors: Tim Boretius, Freiburg (DE); Patrick Kiele, Freiburg (DE)

(21) Appl. No.: 19/108,525

(22) PCT Filed: Sep. 4, 2023
(86) PCT No.: PCT/EP2023/074172

§ 371 Date: Mar. 4, 2025

(30) Foreign Application Priority Data
Sep. 15, 2022 (DE) 10 2022 123 560.2

(51) Int. Cl. A61N 1/375

(52) U.S. Cl. CPC A61N 1/3752

(57) ABSTRACT

An implantable electrical contact arrangement has at least one electrode element arrangement enclosed entirely by biocompatible, electrically insulating material, and an electrode surface that is directly or indirectly enclosed by the biocompatible, electrically insulating material, the electrode element arrangement having a stack-shaped layer composite which has a metallization layer at least on a ceramic substrate over an adhesion promoter layer. The layer composite is bonded to a passivation layer such that covalent bonds are formed, and is otherwise completely encased by the passivation layer, except for at least one surface area of a metallization layer facing away from the layer composite. The passivation layer has a surface facing away from the layer composite and to which the biocompatible, electrically insulating material is directly or indirectly adjacent. The passivation layer is deposited on the layer composite by plasma-enhanced chemical vapor deposition, physical vapor deposition, or wet chemical coating.

### CROSS-REFERENCE TO RELATED APPLICATIONS

This application is the United States national stage entry of International Application No. PCT/EP2023/074172, filed on Sep. 4, 2023, and claims priority to German Application No. 10 2022 123 560.2, filed on Sep. 15, 2022. The contents of International Application No. PCT/EP2023/074172 and German Application No. 10 2022 123 560.2 are incorporated by reference herein in their entireties.

### FIELD

Described is an implantable electrical contact arrangement which has at least one electrode element arrangement enclosed entirely by biocompatible, electrically insulating material, and comprising at least one electrode surface that is directly or indirectly enclosed by the biocompatible, electrically insulating material, the electrode element arrangement having a stack-shaped layer composite which has a metallization layer at least on a ceramic substrate over an adhesion promoter layer.

### BACKGROUND

A particular challenge in the production and design of implantable electrical contact arrangements, via which electrical leads or lead structures are electrically contacted with each other, is the prevention of the penetration of water or moisture into and/or through the material interfaces contained within an electrical contact arrangement. Contacts with water on electrically conducting lead and electrode structures mostly made of metallic material, result in irreversible manifestations of degradation and an associated impairment of the electrical energy and signal transmission properties. Furthermore, a permanently moist environment leads to detachment manifestations between the metallic structures contained within the electrical contact arrangement and the immediately surrounding surfaces, usually made of polymer materials, through which the service life of such contact arrangements is ultimately reduced.

The article by Patrick Kiele et al. “Dünnschicht-Metallisierungsstapel dienen als zuverlässige Leiter auf keramikbasierten Substraten für aktive Implantate” [Thin layer metallization stacks act as reliable conductors on ceramic-based substrates for active implants], IEEE Transactions on components, packing and manufacturing technology, vol. 10, no. 11, November 2020, states that in the manufacturing of medical implants with electrical connecting or contacting structures that are produced by way of conventional metal vapor deposition processes, for example sputter processes, on a ceramic substrate, preferably in the form of an Al₂O₃ substrate, better results can be achieved in terms of adhesive strength, then with a screen printing process using metal paste with a subsequent high temperature process. In particular, good results are achieved in the production of such electrical contact structures through metallization with sputtered aluminium on an adhesive layer of tungsten-titanium which can increase the adhesive strength on the aluminium oxide substrate. Biocompatible, electrically insulating material is then poured around the contact arrangement produced in this way, protecting the inside contact arrangement from the intracorporeal moist environment.

Document DE 10 2016 222 710 A1 discloses an implantable electrical contact arrangement that comprises an electrode element arrangement otherwise entirely enclosed by electrically insulating material, with at least one freely accessible electrode surface that is directly or indirectly enclosed by the biocompatible, electrically insulating material.

### SUMMARY

The object of the present disclosure is to further develop an implantable electrical contact arrangement which has at least one electrode element arrangement enclosed entirely by biocompatible, electrically insulating material, with at least one electrode surface that is directly or indirectly enclosed by the biocompatible, electrically insulating material, the electrode element arrangement having a stack-like layer composite which has a metallization layer at least on a ceramic substrate over an adhesion promoter layer, in such a way that the resistance of the implantable, electrical contact arrangement enclosed by the biocompatible, electrically insulating material is to be significantly improved with regard to moisture or water from the intracorporeal most environment, in order to thereby prolong the life span and the manufacturer-specified maximum intracorporeal operating duration of the medical implant.

Features further expounding the subject matter of the present disclosure are set out in the further description, in particular with reference to the figures.

The implantable electrical contact arrangement according to the present disclosure is characterized in that the stack-shaped layer composite of the electrode element arrangement is bonded to a passivation layer such that covalent bonds are formed, and is otherwise completely encased by said passivation layer, except for at least one surface area of a metallization layer facing away from the layer composite. Moreover, the passivation layer has a passivation layer surface which faces away from the stack-shaped layer composite and to which the biocompatible, electrically insulating material is directly or indirectly adjacent, and like at the sides of the layer composite, forms covalent bonds with the biocompatible, electrically insulating material.

As result of the covalent or chemical bonds forming between the surfaces of the stack-shaped layer composite and the passivation layer, as well as preferably also between the passivation layer and the biocompatible, electrically insulating material as a result of the interaction of the outer electrons between atoms and/or molecules respectively, the penetration of moisture or water at least in the area of the contact arrangement enclosed by the biocompatible, electrically insulating material is at least almost ruled out. In this way, delamination between the boundary surfaces of two adjoining material layers, for example boundary surfaces within the stack-shaped layer composite, but also boundary surfaces between the passivation layer and the stack-shaped composite, as well as between the passivation layer and the surrounding enclosure of biocompatible, electrically insulating material can largely be ruled out.

Like the depositions of the adhesion promoter layer on the ceramic substrate as well as the metallization layer on the adhesion promoter layer, plasma-enhanced chemical vapor deposition (PECVD) is also suitable for the deposition of the passivation layer on the stack-shaped layer composite as well as, if applicable, on available surface areas of the ceramic substrate. Alternatively, also suitable is physical vapor deposition (PVD) or wet chemical coating of the passivation layer on the stack-shaped layer composite, as well as possibly adjoining surface areas of the ceramic substrate.

The passivation layer is provided fully circumferentially on the surface of the stack-shaped layer composite, except at least the surface area of the metallization layer that is provided for electrical contacting with an electrical conductor structure, preferably in the form of an electrical cable, one cable end of which is in permanent firm electrical contact by way of a soldered, bonded or adhesive connection with the surface area of the metallization layer envisaged for contacting.

After producing corresponding electrical contacts on the surface areas of the metallization layer freely accessible for this, the stack-shaped layer composite otherwise completely covered with the passivation layer, along with the ceramic substrate, is enclosed with the biocompatible, electrically insulating material that is preferably made of a polymer and as such in a viscous state can, as part of a pouring process, seamlessly enclose the stack-shaped layer composite, before it transforms into a solid state through setting. Particularly preferred are polymers of the following type: silicones, polyimides, liquid crystal polymers (LCP) or parylene.

The encapsulation formed by solidification of the hardened biocompatible, electrically insulating material also encloses the electrical conductor structures that are in contact with the stack-shaped layer composite.

The electrical conductor structure which is connected to the at least one surface area of the metallization layer in an electrically conducting manner, can in principle have any function and form, for example it can be a separate electrical component which is connected to the metallization layer by way of at least one electrical connection, and with the exception of its electrical connections, is also covered with the passivation layer.

The stack-shaped layer composite is preferably produced in accordance with the design described in the publication cited in the introduction by Kiele et al., i.e. a titanium or tungsten-titanium layer is applied as an adhesion promoter layer on an aluminium oxide layer as a ceramic substrate, onto which in turn a platinum or gold layer is applied in the form of the metallization layer.

In principle, one of the following material compounds is suitable for the passivation layer: silicon oxide, silicon carbide, silicon nitrite or silicon oxinitrite.

### BRIEF DESCRIPTION OF THE DRAWINGS

The present disclosure is described below as an example, without restriction of the general inventive idea by way of examples of embodiment with reference to the drawings.

FIG. 1 shows a perspective view from above of an implantable, electrical contact arrangement; and

FIG. 2 shows a longitudinal section through an implantable contact arrangement in the area of contact with an electrical conductor structure.

### DETAILED DESCRIPTION

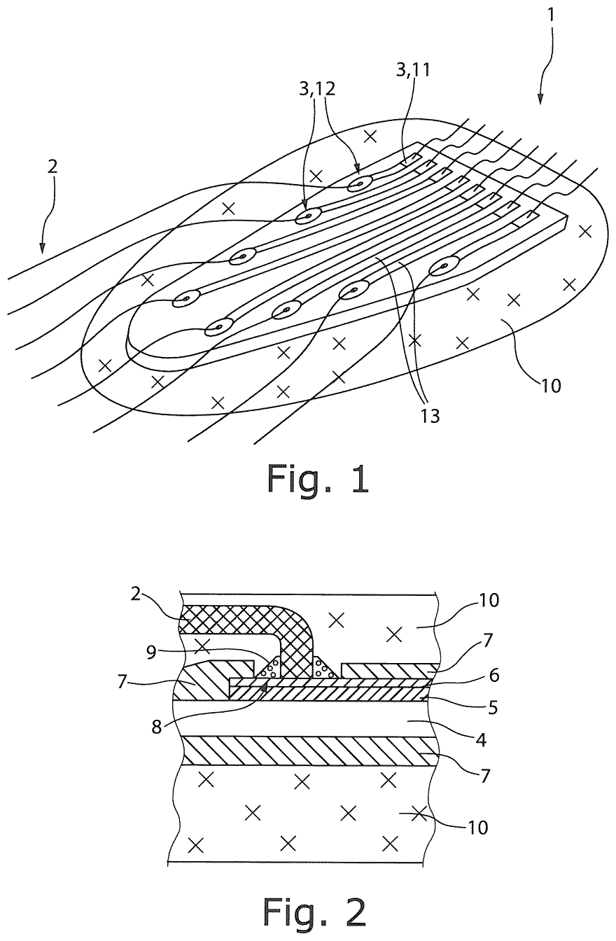

FIG. 1 schematically shows a perspective view of an implantable electrical contact structure designed according to the present disclosure, which is for electrical connection between electrical leads 1 leading to a medically active implant (not shown) with electrical supply and discharge leads 2 respectively that lead to an implantable, not shown supply unit, which provides electrical energy and control signals for the operation of the medical implant.

For the purpose of 1:1 contacting between the electrical leads 1 and the electrical supply and discharge leads 2, there are in each case two, preferably identically configured electrical contact arrangements 3, the layer-shaped structure of which is illustrated in a representative schematic manner in a cross-sectional view in FIG. 2.

On the one hand, the electrical contacts 11 are for electrically contacting the electrical leads 1, and the electrical contacts 12 are for electrically contacting the electrical supply and discharge leads 2. The individual electrical contacts 11 and 12 are each electrically connected to each other in pairs with a metallization layer designed in the form of a strip conductor 13.

By means of physical vapor deposition (PVD), on a ceramic substrate 4, preferably made of Al₂O₃, an adhesion promoter layer 5, preferably mad of WTi or Ti, is deposited, on the surface of which, also using a PVD coating process, a metallization layer 6, preferably in the form of platinum or gold is deposited.

The deposition process for forming the metallization layer 6 takes place using suitable structure masks in order to be able to carry out in this way the minimally dimensioned and geometrically limited layer depositions on the Al₂O₃ substrate 4.

Subsequently, also by means of PVD deposition. a passivation layer 7 is applied both to the surface of the metallization layer 6 as well as the surrounding surface areas of the ceramic substrate 4. Suitable as a passivation layer 7 is preferably silicon oxide, which can form covalent bonds both with the metallization layer 6 and also the ceramic layer 4. With regard to the passivation layer 7, only those surface areas of the metallization layer 6 remain uncovered on which electrical contacting with respectively a supply and discharge lead 2 or an electrical lead 1 is subsequently envisaged.

In FIG. 2, the surface area 8 of the metallization layer 6 envisaged for the electrical contacting of an electrical supply and discharge lead 2, is not covered by the passivation layer 7. The end of the supply and discharge lead 2 directly contacts the surface area 8 of the metallization layer 6 envisaged for producing the contacting and is firmly connected thereto electrically and mechanically by means of a soldered, bonded or adhesive connection 9.

For preferably complete enclosure or encapsulation of the implantable electrical contact arrangement 3, a biocompatible, electrically insulating matrial 10 is applied onto the suface of the passivation layer 7, preferably by means of a pouring process, and forms covalent bonds therewith. In addition, the biocompatible, electrically conductive material 10 also encloses the electrical contact area 3 as well as, at least in the case of the electrical contacts 12, the electrical supply and discharge leads 2, which through the biocompatible, electrically conductive material 10 are mutually electrically insulated and also mechanically stably held, in a similar manner to a matrix directly surrounding the electrical supply and discharge leads 2.

Serving the purpose of the electrical connetion between the electrical contacts 11 and 12 is the metallization layer 6 which is produced between the contact points in each case in the form of separate strip conductors and which locally connects the contact points 11, 12 in pairs. With the exception of the surface areas 8 of the metallization layer 6 envisaged for contacting the electrical cables 1 as well as the electrical supply and discharge leads 2 at the electrical contacts 11 and 12, the passivation layer 7 is deposited over the entire surface on upper side and underside of the ceramic substrate 4 as well as on the strip conductors 13 in order to thus create a uniform surface for forming covalent bonds with the biocompatible and electrically insulating material layer 10 made of polymer material applied above it.

### LIST OF REFERENCE NUMBERS

1 Electrical lead/cable
2 Electrical supply and discharge lead
3 Electrical contact arrangement
4 Ceramic substrate.
5 Adhesion promoter layer
6 Metallization layer
7 Passivation layer
8 Surface area of the metallization layer
9 Soldered, bond or adhesive connection
10 Biocompatible, electrically insulating material
11 Electrical contact
12 Electrical contact
13 Strip conductor

1.-14. (canceled)

15. An implantable electrical contact arrangement which has at least one electrode element arrangement enclosed entirely by biocompatible, electrically insulating material, with at least one electrode surface that is directly or indirectly enclosed by the biocompatible, electrically insulating material, the at least one electrode element arrangement having a layer composite which is stack-shaped and has a metallization layer at least on a ceramic substrate over an adhesion promoter layer, wherein the layer composite is bonded to a passivation layer such that covalent bonds are formed, and is otherwise completely encased by said passivation layer, except for at least one surface area of the metallization layer facing away from the layer composite,
wherein the passivation layer has a passivation layer surface which faces away from the layer composite and to which the biocompatible, electrically insulating material is directly or indirectly adjacent, and
wherein the passivation layer is deposited on the layer composite by plasma-enhanced chemical vapor deposition, physical vapor deposition or wet chemical coating.

16. The implantable electrical contact arrangement according to claim 15, wherein the at least one surface area of the metallization layer not covered by the passivation layer equates to at least one electrode surface area, on which an electrical lead or an electrical supply and discharge lead is contacted.

17. The implantable electrical contact arrangement according to claim 16, wherein electrical contacting of the electrical lead or the electrical supply and discharge lead is implemented based on a soldered, bonded or adhesive connection.

18. The implantable electrical contact arrangement according to claim 15, wherein the metallization layer is a platinum or gold layer.

19. The implantable electrical contact arrangement according to claim 15, wherein the adhesion promoter layer is a tungsten-titanium or titanium layer.

20. The implantable electrical contact arrangement according to claim 15, wherein the passivation layer is a silicon oxide, silicon carbide, silicon nitrite or silicon oxinitrite layer.

21. The implantable electrical contact arrangement according to claim 15, wherein the biocompatible, electrically insulating material is a polymer.

22. The implantable electrical contact arrangement according to claim 21, wherein the polymer is one of: silicones, polyimide, liquid crystal polymer, and parylene.

23. The implantable electrical contact arrangement according to claim 15, wherein an electrical component is connected to the metallization layer by at least one electrical connection, the electrical component being covered with the passivation layer.

24. The implantable electrical contact arrangement according to claim 15, wherein the biocompatible, electrically insulating material is selected in such a way that the biocompatible, electrically insulating material is connected at least to the passivation layer by way of covalent bonds.

25. The implantable electrical contact arrangement according to claim 15, wherein the layer composite is bonded to a passivation layer such that exclusively covalent bonds are formed.

26. The implantable electrical contact arrangement according to claim 15, wherein the passivation layer surface is bonded to the biocompatible, electrically insulating material such that exclusively covalent bonds are formed.

27. The implantable electrical contact arrangement according to claim 15, wherein the ceramic substrate comprises an oxide ceramic.

28. The implantable electrical contact arrangement according to claim 27, wherein the oxide ceramic is Al₂O₃ or ZrO₂.
